(19) United States
(12) Patent Application Publication
(10) Pub. No.: US 2022/0211322 A1
(43) Pub. Date: Jul. 7, 2022

(54) DETECTION OF CHANGES IN PATIENT HEALTH BASED ON SLEEP ACTIVITY

(71) Applicant: Medtronic, Inc., Minneapolis, MN (US)

(72) Inventors: Bruce D. Gunderson, Plymouth, MN (US); Andrew Radtke, Minneapolis, MN (US); Brian B. Lee, Golden Valley, MN (US)

(21) Appl. No.: 17/142,972

(22) Filed: Jan. 6, 2021

(51) Int. Cl. A61B 5/00; A61B 5/11

(52) U.S. Cl. CPC A61B 5/4815; A61B 5/1118; A61B 5/4809; A61B 5/318; A61B 5/7221; A61B 5/6802; A61B 5/7264

(57) ABSTRACT

This disclosure is directed to systems and techniques for detecting changes in patient health based upon monitoring patient sleep activities. One example medical system comprises one or more sensors configured to sense patient activity; sensing circuitry configured to provide patient activity data based on the sensed patient activity; and processing circuitry configured to: determine, from the patient activity data, for each of a plurality of intervals, a respective activity classification, wherein each activity classification indicates whether the patient activity data during the interval satisfies at least one predetermined criterion indicative of patient movement; for each of a plurality of timeslots, determine a number of intervals that satisfy the at least one predetermined criterion, each timeslot including a consecutive subset of the plurality of intervals; and identify transitions between an inactive state and an active state of the patient based on the determined numbers of intervals within the plurality of timeslots.

### FIELD

The disclosure relates generally to medical systems and, more particularly, medical systems configured to monitor patient sleep history for changes in patient health.

### BACKGROUND

Some types of medical systems may monitor various data (e.g., a cardiac electrogram (EGM) and activity) of a patient or a group of patients to detect changes in health. In some examples, the medical system may monitor the cardiac EGM to detect one or more types of arrhythmia, such as bradycardia, tachycardia, fibrillation, or asystole (e.g., caused by sinus pause or AV block). In some examples, the medical system may include one or more of an implantable or wearable medical device or other devices to collect various measurements used to detect changes in patient health. In some examples, the medical system may analyze measurements recorded over a period of time for trends that may indicate certain changes in patient health.

### SUMMARY

Medical systems and techniques as described herein detect changes in health for a patient based upon that patient's sleep activity over short time periods (e.g., one or a few days) or longer time periods. Sleep, in general, plays a significant role in any patient's health by supporting a considerable number of other patient activities. Additionally, other maladies may cause a change in a patient's sleep pattern. An example patient (over at least one day) may exhibit an unhealthy or concerning sleeping pattern, for example, when that patient's (recent) sleep pattern deviates from a pre-determined baseline and/or from the patient's own sleep history. By determining that the patient's sleep pattern is abnormal, medical systems and techniques described herein may warn the patient, a caregiver, or a clinician, and, possibly, allow an intervention to improve sleep or an underlying condition affecting sleep.

In some examples, medical systems and techniques described herein enable the detection of changes in patient health by implementing a medical device positioned on the thorax or core of the patient to accurately identify sleep and awake periods of the patient's (daily) activities. By doing so, these medical systems and techniques may avoid false determinations caused by noisy device measurements (e.g., associated with wrist or extremity worn patient activity monitors) and/or patient manipulation of the device (e.g., removing the device from the patient).

A variety of medical devices (e.g., implantable devices, wearable devices, etc.) may be configured to monitor patient activity data, detect time periods where the patient is a inactive state or an active state based on the activity data, and detect changes in the patient's health that correlate to changes in data recording the patient's sleep activity each day over a number of days. As demonstrated herein, these changes may be in reference to a pre-determined baseline of (e.g., generic) healthy patient activity data or the patient own sleep history. When people in general experience a change in health, cotemporaneous abnormal or irregular sleep patterns are often observed. In addition, inactive and active states (e.g., over twenty-four hour days) of the patient's activities (e.g., aggregated over a number of days) may correlate with an accurate assessment of the patient's health and monitoring those time periods may provide an enhanced indication of changes in the patient's health.

In one example, a medical system comprises one or more sensors configured to sense patient activity; sensing circuitry configured to provide patient activity data based on the sensed patient activity; and processing circuitry configured to: determine, from the patient activity data, for each of a plurality of intervals, a respective activity classification, wherein each activity classification indicates whether the patient activity data during the interval satisfies at least one predetermined criterion indicative of patient movement; for each of a plurality of timeslots, determine a number of intervals that satisfy the at least one predetermined criterion, each timeslot including a consecutive subset of the plurality of intervals; and identify transitions between an inactive state of the patient and an active state of the patient based on the determined numbers of intervals within the plurality of timeslots.

In another example, a method comprises sensing patient activity via one or more sensors; generating, via sensing circuitry, patient activity data based on the sensed patient activity; from the patient activity data, determining, by processing circuitry, for each of a plurality of intervals, a respective activity classification, wherein each activity classification indicates whether the patient activity data during the interval satisfies at least one predetermined criterion indicative of patient movement; for each of a plurality of timeslots, determining, by the processing circuitry, a number of intervals that satisfy the at least one predetermined criterion, each timeslot including a consecutive subset of the plurality of intervals; and identifying, by the processing circuitry, transitions between an inactive state of the patient and an active state of the patient based on the determined numbers of intervals within the plurality of timeslots.

In another example, a non-transitory computer-readable storage medium comprises program instructions that, when executed by processing circuitry of a medical system, cause the processing circuitry to: sense patient activity via one or more sensors; generate, via sensing circuitry, patient activity data based on the sensed patient activity, determine, from the patient activity data, for each of a plurality of intervals, a respective activity classification, wherein each activity classification indicates whether the patient activity data during the interval satisfies at least one predetermined criterion indicative of patient movement; for each of a plurality of timeslots, determine a number of intervals that satisfy the at least one predetermined criterion, each timeslot including a consecutive subset of the plurality of intervals; and identify transitions between an inactive state of the patient and an active state of the patient based on the determined numbers of intervals within the plurality of timeslots.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a functional block diagram illustrating an example configuration of a medical device, in accordance with one or more examples of the present disclosure.

FIG. 3 is a conceptual side-view diagram illustrating an example configuration of the IMD of FIGS. 1 and 2, in accordance with one or more examples of the present disclosure.

FIG. 4 is a functional block diagram illustrating an example configuration of the external device of FIG. 1, in accordance with one or more examples of the present disclosure.

FIG. 5 is a block diagram illustrating an example system that includes an access point, a network, external computing devices, such as a server, and one or more other computing devices, which may be coupled to the medical device and external device of FIGS. 1-4, in accordance with one or more examples of the present disclosure.

FIG. 6 is a flow diagram illustrating an example operation for monitoring patient activity to detect transitions between inactive and active state and determine sleep quality metrics to enable detection of changes in patient health, in accordance with one or more examples of the present disclosure.

FIG. 7 is a flow diagram illustrating an operation for determining, for each interval, a respective activity count classification, in accordance with one or more examples of the present disclosure.

FIG. 8 is a state diagram illustrating an example technique for identifying transitions between inactive state and active state based on activity data over time, in accordance with one or more examples of the present disclosure.

Like reference characters denote like elements throughout the description and figures.

### DETAILED DESCRIPTION

In general, medical systems according to this disclosure implement techniques for detecting changes in patient health based upon patient sleep metric values. Some techniques determine (e.g., detect) exactly when the patient is in an inactive state or an active state and use those determinations to provide the accurate patient sleep metric values. During the inactive state, the patient is most likely asleep for a length of time (e.g., a sleep period). During the active state, the patient may be awake for a length of time (e.g., an awake period) while in other instances the patient may be awake for a trivial length of time before going back to sleep. In some examples, when the patient falls back asleep within a configurable time period (e.g., after onset of an out-of-bed event), the patient may be in an active state but is not considered awake during that time period and the patient's sleep period continues to toll. If the patient does not fall back asleep within the configurable time period (e.g., after end of out-of-bed event) and exhibits high activity levels and/or a certain time of day is reached (e.g., 8 am), the patient's sleep period ends (e.g., sleep-offset) and the patient is considered awake after having successfully transitioned to an active state. The patient's awake period tolls until onset of a next sleep period. Some techniques described herein determine accurate times (e.g., timestamps) for the patient's sleep and awake periods by determining transitions between inactive and active states, including out-of-bed events and sleep-onset events.

The patient's activity data (e.g., activity counts) provides highly accurate data for computing an abstraction (e.g., a sleep quality metric value) to represent the recorded patient sleep activity. The techniques described herein include performing a detection analysis to detect changes in patient health by detecting inactive and active states including events that transition the patient between these states. In one example, the techniques described herein utilize activity counts and/or activity count classifications to determine whether the patient's current activity data indicates too much activity for the patient to be asleep and if so, the patient is determined to be in the active state, but if the patient is sufficient inactive to be asleep, the patient is determined to be in the inactive state. The patient's activity counts over a plurality of timeslots (e.g., one (1) minute timeslots) may be indexed by timeslot order and, in one example, the techniques described herein compare a set of activity count classifications for a consecutive subset of timeslots to predetermined criterion indicative of patient movement. Based on such predetermined criterion, the techniques described herein enable a medical system to accurate determine whether the patient's (recent) activity level indicates the inactive state or the active state. In some examples, the medical system may improve upon the accuracy of the techniques described herein by adjusting the predetermined criterion dynamically based on performance or based on patient or physician input.

Such a comparison not only simplifies the detection analysis, for example, by leveraging activity sensors (e.g., accelerometers) an implanted sensor (e.g., biosensor) and relying only on the patient activity data (e.g., from accelerometers), but also improves upon sensitivity and specificity of that detection analysis. For at least these reasons, some medical systems and techniques described herein do not utilize a wearable device or any external sensors for the patient activity data, lowering operational resource requirements and costs. Medical systems described herein may restrict the detection analysis to a limited amount of available patient activity data and still achieve a high level of accuracy when detecting transitions between inactive and active states. An example medical system may also subject the analysis to resource limitations (e.g., a limited memory capacity such as a maximum buffer size) while maintaining the high level of accuracy. Due to the above limitations, the techniques described herein may require fewer criteria for the detection analysis to accurately identify with the patient activity data indicates a transition from the active state to the inactive state and vice versa. The techniques allow the detection analysis to operate without requiring sensor data other than activity data (e.g., from an accelerometer) as other types of sensor data may be unavailable. A medical device equipped with substantial resource capacities and sensing capabilities is not needed; instead, a medical device with fewer capacities of processing, network, and storage resources can be configured to detect changes in patient health in accordance with any technique described herein.

Example medical devices that may collect patient activity data may include an implantable or wearable monitoring device, a pacemaker/defibrillator, or a ventricular assist device (VAD). An implantable or wearable device with an accelerometer continuously measuring the patient activities and recording such measurements as patient activity data is described herein as example medical devices. One example medical device implements a system and technique directed to detect inactive and active states based on a number of activity count classifications within a timeslot encompassing one or more (consecutive) time intervals.

The example medical device may communicate the patient activity data to other devices, such as a computing device, and those devices may further analyze the patient activity data and then, provide a report regarding the patient's activities and health. The report may compare various implementations of the techniques described herein, for example, comparing, for the same patient, respective sleep quality metric values provided by different selections of inactive and active states. Sleep quality metric values based on activity data during active and inactive states may provide a patient or caregiver information an important aspect of the patient's health.

In this manner, the techniques of this disclosure may advantageously enable improved accuracy in the detection of changes in patient health and, consequently, better evaluation of the condition of the patient.

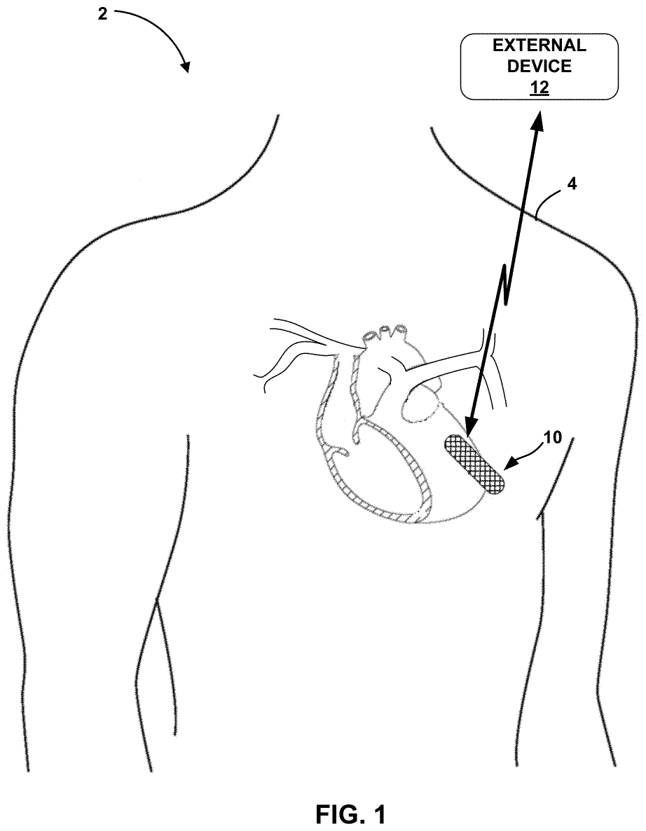

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac EGM via the plurality of electrodes. In some examples, IMD 10 takes the form of the LINQ™ ICM available from Medtronic, Inc. of Minneapolis, Minn. IMD 10 includes one or more sensors configured to sense patient activity, e.g., one or more accelerometers.

External device 12 may be a computing device with a display viewable by the user and an interface for receiving user input to external device 12. In some examples, external device 12 may be a notebook computer, tablet computer, workstation, one or more servers, cellular phone, personal digital assistant, or another computing device that may run an application that enables the computing device to interact with IMD 10.

External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

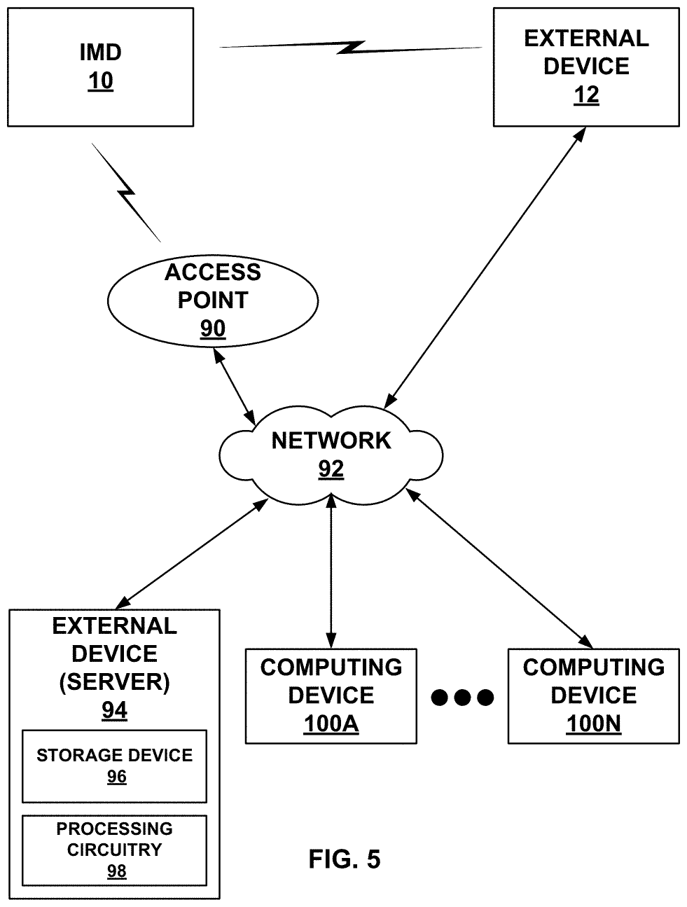

External device 12 may be used to configure operational parameters for IMD 10. External device 12 may be used to retrieve data from IMD 10. The retrieved data may include values of physiological parameters measured by IMD 10, indications of episodes of arrhythmia or other maladies detected by IMD 10, and physiological signals recorded by IMD 10. For example, external device 12 may retrieve cardiac EGM segments recorded by IMD 10 due to IMD 10 determining that an episode of asystole or another malady occurred during the segment. As another example, external device 12 may receive patient activity data, sleep quality metric values, or other data related to the techniques described herein from IMD 10. As will be discussed in greater detail below with respect to FIG. 5, one or more remote computing devices may interact with IMD 10 in a manner similar to external device 12, e.g., to program IMD 10 and/or retrieve data from IMD 10, via a network.

Processing circuitry of medical system 2, e.g., of IMD 10, external device 12, and/or of one or more other computing devices, may be configured to perform the example techniques of this disclosure for detecting transitions between inactive and active states to detect changes in patient health. Processing circuitry of IMD 10 may be communicably coupled to one or more sensors, each being configured to sense patient physiological parameters in some form (e.g., patient activity including generic patient movements), and sensing circuitry configured to generate sensor data (e.g., patient activity data). For example, processing circuitry of IMD 10 may be communicably coupled to one or more accelerometers of which each may be configured to sense patient activity in general, and the sensing circuitry configured to generate the patient activity data.

Processing circuitry of IMD 10, possibly in combination with processing circuitry of external device 12, may compute sleep quality metric values from the patient activity data and after a number of days, analyze the metric values for indicia of patient health including non-trivial changes in patient health. To facilitate a successful analysis, the processing circuitry may identify inactive and active states (e.g., during a pre-configured time of day such as between 9 pm and 6 am) to be used in the computation of the daily values. Although described in the context of examples in which IMD 10 that senses patient activity comprises an insertable cardiac monitor, example systems including one or more implantable, wearable, or external devices of any type configured to sense patient activity may be configured to implement the techniques of this disclosure.

In some examples, processing circuitry in a wearable device may execute same or similar logic as the logic executed by processing circuitry of IMD 10 and/or other processing circuitry as described herein. In this manner, a wearable device or other device may perform some or all of the techniques described herein in the same manner described herein with respect to IMD 10. In some examples, the wearable device operates with IMD 10 and/or external device 12 as potential providers of computing/storage resources and sensors for monitoring patient activity and other patient parameters. For example, the wearable device may communicate the patient activity data to external device 12 for storage in non-volatile memory and for computing sleep quality metric values from peak patient activity data and non-peak patient activity data. Similar to processing circuitry of IMD 10, processing circuitry of external device 12 may analyze the patient activity data to determine which peak and non-peak periods to use in computing the sleep quality metric values.

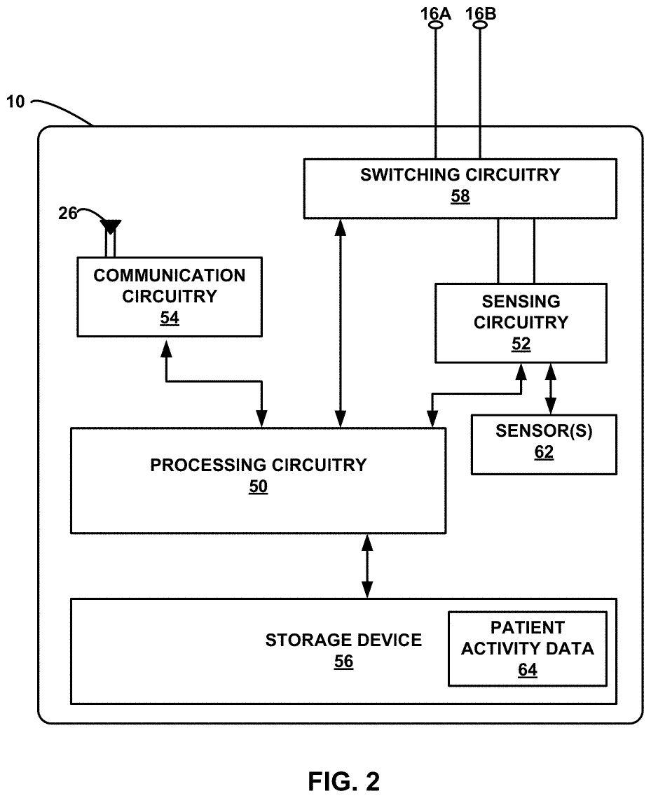

FIG. 2 is a functional block diagram illustrating an example configuration of IMD 10 of FIG. 1 in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to sense electrical signals of the heart of patient 4, for example by selecting the electrodes 16 and polarity, referred to as the sensing vector, used to sense a cardiac EGM, as controlled by processing circuitry 50. Sensing circuitry 52 may sense signals from electrodes 16, e.g., to produce a cardiac EGM, in order to facilitate monitoring the electrical activity of the heart. Sensing circuitry 52 may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62. Sensing circuitry 52 may capture signals from any one of sensors 62, e.g., to produce patient activity data 64, in order to facilitate monitoring the patient activity and detecting changes in patient health.

Sensing circuitry 52 may generate patient activity data 64 from sensor signals received from sensor(s) 62 that encode the patient activity and other physiological parameters. Sensing circuitry 52 and processing circuitry 50 may store the patient activity data 64 in storage device 56.

Processing circuitry 50, executing logic configured to perform a detection analysis on the sensor data, is operative to detect any change (e.g., a decline) in patient health. Processing circuitry 50, via sensing circuitry 52, may control one or more of sensors 62 to sense patient activity in some form; examples of sensors 62 to sense patient activity include an accelerometer (e.g., a three-axis accelerometer), a gyroscope, a temperature gauge, a moment transducer, and/or the like. There are a number of methods for converting the patient activity data into activity levels, which may be a quality (e.g., high activity, low activity, and/or the like) or a quantity (e.g., a number of threshold crossings or sum of activity signal samples per time interval, such as a 10-second interval, which are sometimes referred to as counts or activity counts). Activity levels (e.g., activity counts) enable differentiation between multiple timeslots (e.g., two or more consecutive intervals, two or more non-consecutive intervals, two or more groups of contiguous intervals and/or the like) of patient activity data 64 in terms of actual patient movement.

In performance of the above detection analysis, processing circuitry 50 converts activity counts into an activity classification for an interval (e.g., a ten (10) second interval) encompassing a plurality of sub-intervals (e.g., two (2) second sub-intervals). In some examples, processing circuitry 50 is configured to determine an activity classification for each interval by evaluating sensed patient activity using any suitable criterion (e.g., pre-determined criterion indicative of patient movement (i.e., an active state)). In general, processing circuitry 50 checks whether counts of a frontal (z-axis) accelerometer reach or exceed a noise floor threshold within each of consecutive 10-second time windows referred to herein as intervals. Processing circuitry 50 stores a limited amount of data associated with 10-second activity intervals where any interval for which activity reaches or exceeds the noise floor threshold counts as an activity classification. It is noted that activity classification may be alternatively referred to herein as an activity count classification or an awake classification. Combining the classifications for multiple consecutive activity intervals in a timeslot results in a number of activity count classifications for the timeslot; for example, each activity count classification may represent ten-seconds, and six (e.g., consecutive) intervals may represent a minute. The number of intervals in which the noise floor threshold was satisfied for a timeslot, e.g., minute, may be stored, e.g., in a memory buffer in storage device 56.

Processing circuitry 50 of IMD 10 (and/or processing circuitry of another device of system 2, such as external device 12 or a networked computing device) may execute logic programmed with any example sleep quality metric; while under the control of the executed logic, processing circuitry 50 of IMD 10 applies a corresponding formula to a particular day's sleep and (possibly) awake patient activity data and computes the sleep quality metric value. The processing circuitry may determine sleep quality metrics based on the times at which transitions from the active state to the inactive state and vis-a-versa occur. Example sleep quality metrics include total sleep duration per day, number of arousals per day, length of longest uninterrupted sleep period per day, duration of sleep during a certain period or periods of the day such as nighttime, among other variables.

Processing circuitry 50 of IMD 10 (and/or processing circuitry of another device of system 2, such as external device 12 or a networked computing device) tracks the sleep quality metric values over time to detect changes in patient health. In one example, processing circuitry 50 compares two or more sleep quality metric values to each other and identifies at least one difference worth analyzing for any indication of a change in patient health. Over time, the example sleep quality metric values may form a range and that range corresponds to a baseline representing normal patient health. Deviations from the range that are either statistically significant or otherwise noteworthy indicate a change in patient health. If the patient is known to be healthy, processing circuitry 50 of IMD 10 may establish the range as a healthy baseline such that substantial (negative) deviations from the range may indicate a decline a patient health. Substantial or otherwise noteworthy deviations include deviations from the established baseline that violate a policy, exceed a threshold difference, cross a maximum activity value, and/or the like. It is noted that other activity metrics may implement different formulas, etc., and the present disclosure does not limit the application to any one activity metric.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include patient activity data 64, activity classifications, numbers of activity classifications, times of transitions between inactive and active states, sleep quality metric values and/or indications of changes in patient health including indications of satisfaction of various sleep criteria.

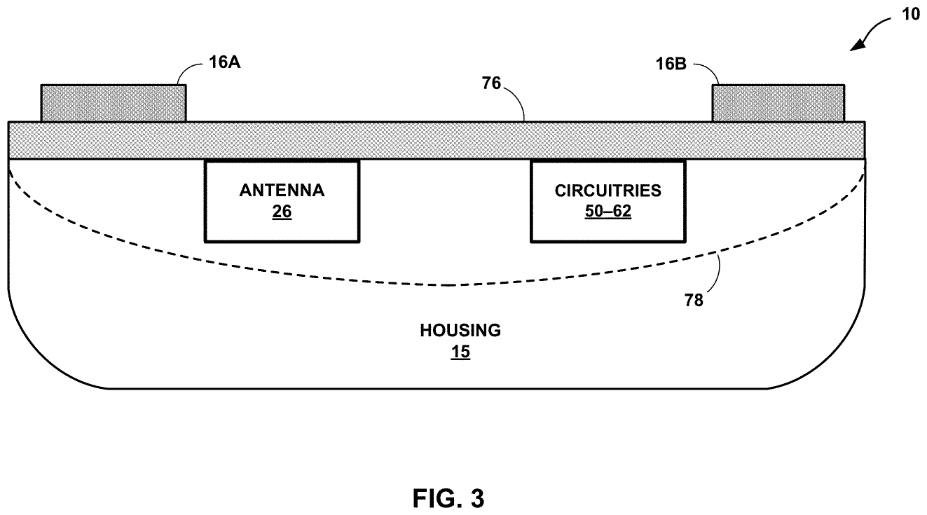

FIG. 3 is a conceptual side-view diagram illustrating an example configuration of IMD 10 of FIGS. 1 and 2. While different examples of IMD 10 may include leads, in the example shown in FIG. 3, IMD 10 may include a leadless, subcutaneously-implantable monitoring device having a housing 15 and an insulative cover 76. Electrode 16A and electrode 16B may be formed or placed on an outer surface of cover 76. Circuitries 50-62, described above with respect to FIG. 2, may be formed or placed on an inner surface of cover 76, or within housing 15. In the illustrated example, antenna 26 is formed or placed on the inner surface of cover 76, but may be formed or placed on the outer surface in some examples. In some examples, insulative cover 76 may be positioned over an open housing 15 such that housing 15 and cover 76 enclose antenna 26 and circuitries 50-62, and protect the antenna and circuitries from fluids such as body fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

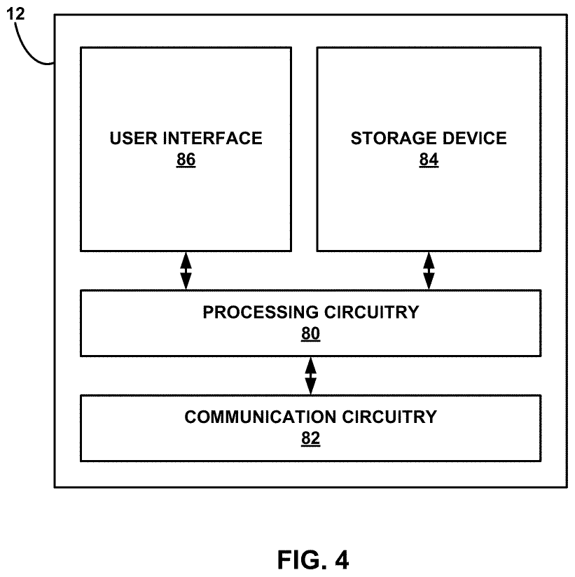

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC, RF communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., patient activity data) to external device 12. In turn, external device 12 may receive the collected data from IMD 10 and store the collected data in storage device 84. The data external device 12 receives from IMD 10 may include sensor data including patient activity data 64. Processing circuitry 80 may implement any of the techniques described herein to analyze data from IMD 10, e.g., activity data 64, to detect sleep onset and offset, and/or analyze the times of transitions between inactive and active states to determine sleep quality metric values e.g., to determine whether the patient is experiencing a change in health based upon one or more criteria.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., sleep quality metric values, indications of changes in sleep quality metric values, and indications of changes in patient health that correlate to the changed in sleep quality metric values, determinations of probability data of possible medical conditions based on the changes in patient health, and/or the like. In addition, user interface 86 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 5 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 5, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as a patient's activity data 64, data indicating activity count classifications for a plurality of time intervals, data indicating times of transitions between inactive and active states, indications of sleep quality metric values, and/or indications of changes in patient health, to access point 90. IMD 10 may partition the patient activity data into separate datasets for inactive state and active state activities prior to transmission of that data. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic CareLink® Network.

In some examples, one or more of computing devices 100 may be a tablet or other smart device located with a clinician, by which the clinician may program, receive alerts from, and/or interrogate IMD 10. For example, the clinician may access data (e.g., patient activity data, inactive and active state transition data, sleep quality metric values, respective activity count classifications for a plurality of time intervals and/or indications of patient health) collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 94 includes a storage device 96, e.g., to store data retrieved from IMD 10, and processing circuitry 98. Although not illustrated in FIG. 5 computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 100 may implement any of the techniques described herein to analyze information, e.g., data 64 received from IMD 10, e.g., to determine whether the health status of a patient has changed, to determine whether prediction criteria are satisfied and/or false prediction criteria are satisfied.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

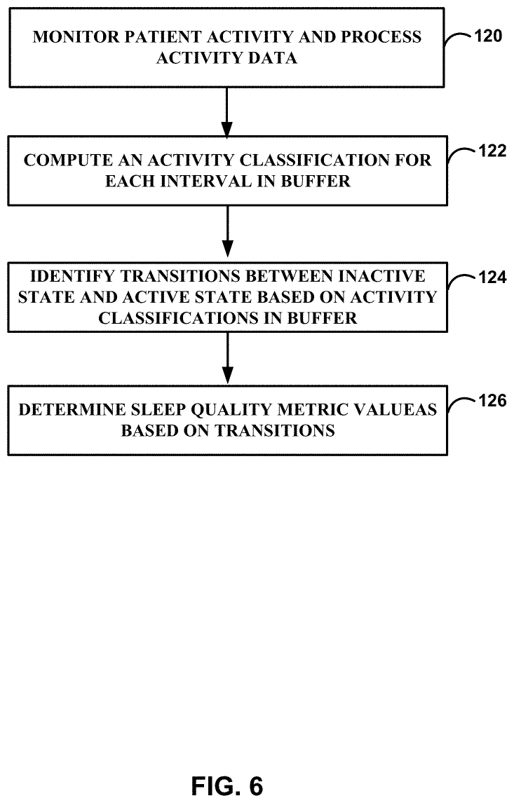

FIG. 6 is a flow diagram illustrating an example operation for monitoring patient sleep quality to detect potential changes in patient health, in accordance with one or more examples of the present disclosure. In some examples, a medical device includes computing hardware operative to execute the example operation. According to the illustrated example of FIG. 6, processing circuitry 50 of IMD 10 monitors patient activity data generated by sensing circuitry 52 of IMD 10 (120). For example, as discussed in greater detail with respect to FIGS. 1-2, processing circuitry 50 may monitor the patient activity data over a number of days and initiates, each day, a method to detect inactive and active states of the patient activity data for computing sleep quality metric values.

In the example operation of FIG. 6, it is noted that there is a range of pre-determined options that may be programmed into examples of IMD 10: A number of time periods in a day (e.g., 24 hour or 48 half-hour time periods) or a length of a given time period (e.g., a half-hour), a number of time intervals in a time slot, a number of time slots in a time period, a maximum buffer capacity (e.g., an amount of a data for a subset of the number of time slots), a selection of a statistical process to apply, a selection regarding which category of patient health to detect, and/or the like.

In the illustrated example, processing circuitry 50 of IMD 10 monitors patient activity and processes activity data (e.g., patient activity data 64) (120). As depicted in FIG. 2, sensor(s) 62 represent devices that sense patient activity including patient movements and generate signals conveying data indicative of the sensed patient activity. Sensing circuitry 52 within IMD 10 captures these signals and generates the activity data being processed by processing circuitry 50.

Processing circuitry 50 computes an activity count classification for each interval in a buffer (122). The buffer may be a portion of storage device 56 and processing circuitry 50 stores, in that buffer, structured datasets of activity data over a pre-defined time period; one example dataset stores, for each timeslot, an activity count classification for each interval in that timeslot. In other examples, the buffer may arrange the activity data into a sequence of 2-second Integrated Counts (ICs) spanning the time period encompassing a number of timeslots. Processing circuitry 50 computes an activity count classification for each ten-second interval in the buffer by combining the 2-second ICs encompassing that ten-second interval into an activity count and comparing that activity count to one or more criterion; if the activity count satisfies the one or more criterion (e.g., meets and/or exceeds a noise floor), processing circuitry 50 sets the activity count classification for that interval at one (1), otherwise the activity count classification for that interval is set to zero (0). A positive activity count classification may be indicative of actual patient movement as opposed to non-zero activity signal (e.g., IC) values being caused by noise and/or other interfering effects. To conserve memory, processing circuitry 50 may create a new dataset for the activity count classifications, overwriting the sequence of 2-second Integrated Counts (ICs), and format each dataset entry as a Boolean value. Hence, the buffer may store TRUE for any positive activity count classification and FALSE for any negative activity count classification because the activity count classification distinguishes valid patient movement from little or no movement for a particular ten-second interval. In some examples, the positive activity count classification signifies that the patient may be awake.

When the buffer has substantially thirty minutes of activity count classifications, processing circuitry 50 may sum the six activity count classifications over the six intervals in each minute long timeslot, which results in 30 integer values for comparison with sleep monitoring criteria. For each positive activity count classification in a given timeslot, processing circuitry 50 increments (by at least one) the corresponding number of activity count classifications for the timeslot.

Processing circuitry 50 identifies transitions between inactive and active states based on activity count classifications in the buffer (124). Given the thirty activity count classifications, processing circuitry 50 may compare one or more classifications with various sleep monitoring criteria and if each sleep monitoring criterion is satisfied, processing circuitry 50 determines that the patient transitioned from inactive state to active state at some point in the thirty minutes or possibly before the thirty minutes. If it is known that the patient was in the inactive state before a first interval in the buffer and, for the earliest intervals, the number of activity count classifications exceeds some threshold, processing circuitry 50 may determine that the patient transitioned to the active state immediately before or contemporaneous with the first interval in the buffer. It is possible that the patient would transition back to the inactive state at some point in the remainder of the thirty minutes (e.g., at the last few intervals in the buffer). For example, if the number of activity count classifications over each minute in the last three minutes in the buffer does not exceeds a threshold, processing circuitry 50 may determine that the patient transitioned to the inactive state.

Vice versa, if it is known that the patient was in the active state before the first interval in the buffer and, for the earliest intervals, there are zero or 1 positive activity count classifications, processing circuitry 50 may determine that the patient transitioned to the inactive state immediately before or contemporaneous with the first interval in the buffer. It is possible that the patient would transition back to the active state at some point in the remainder of the thirty minutes (e.g., at the last few intervals in the buffer). For example, if the number of activity count classifications over each minute in the last three minutes in the buffer exceeds a threshold, processing circuitry 50 may determine that the patient transitioned to the active state. It should be noted that thirty minutes is considered a sufficient amount of time to detect transitions between inactive and active states; however, the present disclosure does not foreclose on alternative lengths of time for the buffer.

Processing circuitry 50 determines sleep quality metric values based on transitions (126). There are a number of metrics (e.g., formulas) for evaluating the patient's activity data (including the activity counts, activity count classifications, and times of transitions between and inactive state and an active state) and any one or more are available to processing circuitry 50 of IMD 10. IMD 10 and external device 12 may cooperate to determines the sleep quality metric values based on identified transitions. In some examples, IMD 10 communicates the buffer (in entirety or a minute at a time) and processing circuitry 80 generates and/or updates the sleep quality metric values for the entire buffer. Processing circuitry 50 may identify transitions and determine metric values in real-time or retroactively. As described herein, processing circuitry 50 and/or processing circuitry 80 may evaluate the sleep quality metric values and determine whether the patient's sleep activity indicates a change in health. According to the present disclosure, processing circuitry 50 of IMD 10 detects changes in patient health based on the sleep quality metric values that are computed from inactive state and active state activity data generated by sensing circuitry 52 of IMD 10 and then, partitioned by processing circuitry 50 of IMD 10.

In the illustrated example of FIG. 6, processing circuitry 50 of IMD 10 leverages the inactive state/active state determinations (e.g., including identified transitions between inactive state and active state) to focus the analysis for detecting changes in patient health on accurate patient activity data. By knowing exactly when the patient became awake or fell asleep, processing circuitry 50 may gain insight into patient's sleeping habits with fewer or no errors. Instead of noise and/or false detections of sleep and awake periods, processing circuitry 50 avails very accurate patient activity data to compute an example abstraction of the patient's sleep activity. Depending upon which activity metrics processing circuitry 50 of IMD 10 employs for the computation, the metric value provides certain insights into the patient's sleep quality. An example metric may refer to mathematical functions (e.g., formulas), data structures (e.g., models), standardized methods/mechanisms, and measurements and other data configured to enable the computation of accurate metric values by way of determining when a patient is awake (i.e., active state) or asleep (i.e., inactive state). Such an analysis may be qualitative and/or quantitative, for example, to gain insight into the patient's health by expressing as many related features as possible given the context of patient sleep activity.

To illustrate by way of an example detection analysis, after computing the sleep quality metric value, processing circuitry 50 of IMD 10 compares the sleep quality metric value with a baseline value. In some examples, the baseline value may be another (e.g., previous) sleep quality metric value representing a highest or average activity metric/level of daily patient sleep quality. Hence, processing circuitry 50 of IMD 10 may compare the computed sleep quality metric value with other sleep quality metric values and then, use the comparison results to detect changes in patient health. If a difference/deviation between the computed sleep quality metric value and the baseline value does not exceed the threshold, processing circuitry 50 of IMD 10 determines no change in patient health. If a difference/deviation between the computed sleep quality metric value and the baseline value exceeds the threshold, processing circuitry 50 of IMD 10 proceeds to generate output data indicating a change in patient health.

In other examples, the baseline value may be pre-determined or, as an alternative, computed through other means than the one or more activity metrics while retaining a same data model to facilitate a comparison with sleep quality metric values. In some examples, the baseline value represents normal health for that specific patient and any deviation from that baseline value should be evaluated. The baseline value may represent the patient's border in that the baseline value is the highest activity value/level while still indicating no decline in that patient's health; any deviation beyond the baseline value may indicate an acute change or decline in the patient's health.

Sleep activity/quality metrics as described herein include any number of formulas, methods, and mechanisms for determining the metric value from available patient activity data. Some activity metrics are configured to exploit features corresponding to overall patient health. Other activity metrics may be configured with a more granular feature set to identify changes in different categories of patient health such as heart health.

Processing circuitry 50 of IMD 10 may apply one or more sleep quality metrics to each day's sleep and awake patient activity data and computes the sleep quality metric values for a number of days. Over time, the example sleep quality metric values may form a range and that range corresponds to a baseline representing normal patient health. Deviations from the range that are either statistically significant or exceed a pre-determined threshold indicate a change in patient health. One example sleep quality metric measures night sleep restlessness as an average of the four lowest activity count classifications during the last 24 hours.

As one example of the output data described above, processing circuitry 50 of IMD 10 may combine two or more metrics for trending and analysis. The sleep quality metric values may be graphed over time to visually see changes and trends. A linear regression line could be displayed for the last 2 weeks to show the general trend. An alert could be provided for an acute decrease in daytime activity or acute change in sleep restlessness using Statistical Process Control (SPC). An alert would indicate a significant decline in health that would need to have further evaluation, which could involve taking a temperature, measuring oxygen, and asking about symptoms from a caregiver or clinician. This and other medical measurements would be used to determine specific causes (e.g. flu, depression).

As a response to detecting the deviation that exceeds the threshold, processing circuitry 50 of IMD 10 may proceed to generate a report describing the patient's health over time. IMD 10 may leverage a remote computing device such as external device 12 for additional processing power to generate the report. IMD 10 may provide external device 12 with the patient activity data over a sufficient number of days (e.g., an experiment period). In turn, processing circuitry 80 of external device 12 may generate the report to include a graph plotting the sleep quality metrics over a sufficient number of days (e.g., an experiment period). Processing circuitry 80 of external device 12 may apply different activity metrics to the same patient activity data for comparison. If IMD 10 is programmed to use fixed 10 second time intervals, remote computing device may generate graphs plotting the sleep quality metrics if individualized time intervals or dynamic time intervals are employed. In this manner, the effectiveness of different techniques may be assessed. Each technique may be applied to an individual or may be group-based.

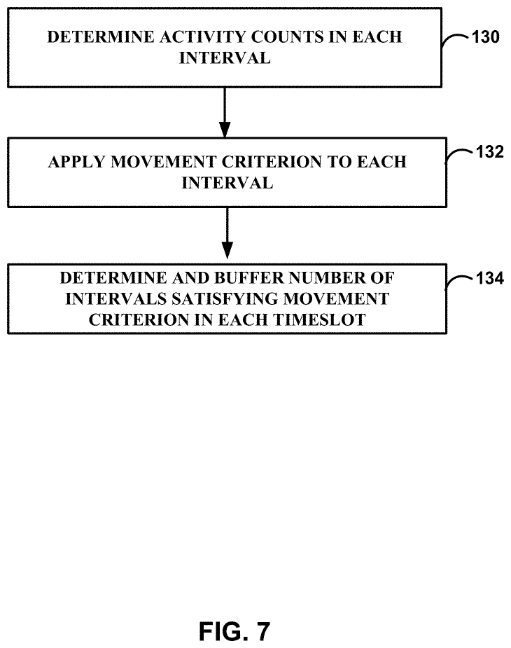

FIG. 7 is a flow diagram illustrating an example operation for computing an activity count classification for each interval in a buffer, in accordance with one or more examples of the present disclosure. In some examples, a medical device includes computing hardware operative to execute the example operation. According to the illustrated example of FIG. 6, processing circuitry 50 of IMD 10 monitors patient activity data generated by sensing circuitry 52 of IMD 10 (120). For example, as discussed in greater detail with respect to FIGS. 1-2, processing circuitry 50 may monitor the patient activity data over a number of days and initiates, each day, a method to detect inactive and active states of the patient activity data for computing sleep quality metric values.

In the illustrated example, processing circuitry 50 of IMD 10 determines a number of activity counts in each interval of each timeslot, which encompasses a number of intervals (e.g., six intervals for a minute long timeslot) (130). As described herein, the buffer may arrange data entries of a dataset where each entry represents a timeslot and stores a number of activity count classifications for that timeslot. To determine the number of activity count classifications for each timeslot, processing circuitry 50 determines the activity counts for each interval in that timeslot.

Processing circuitry 50 applies movement criterion to each interval in the buffer (132). To compute respective activity count classifications for the intervals, processing circuitry 50 compares the corresponding activity count for each time interval to the movement criterion and if the corresponding activity count satisfies the movement criterion, processing circuitry 50 registers an activity count classification for that interval. An example movement criterion is a noise floor that processing circuitry 50 determines lowest level(s) of noise representative of substantially no patient activity (e.g., movement). Processing circuitry 50 or processing circuitry 80 may compute the noise floor by performing the following steps: a) recording at least 24 hours of ICs; b) determine a sum for every five consecutive 2 sec IC (10 seconds); c) select one or more lowest values (e.g. 1 hour); and d) determine a highest of the lowest values as the noise floor. If the activity counts in any interval exceeds the noise floor, one may be confident that the patient was active and moving during the time of that interval.

Processing circuitry 50 determines and buffers a number of time intervals satisfying movement criteria in each timeslot (134). As described herein, processing circuitry 50 stores a number of activity count classifications for each timeslot in the buffer by incrementing the stored number of activity count classifications for each interval satisfying the movement criterion in that timeslot. Once the number of activity count classifications is determined for each timeslot in the buffer, processing circuitry 50 may proceed to identify inactive state/active state transitions (if any) based on the activity count classification sums.

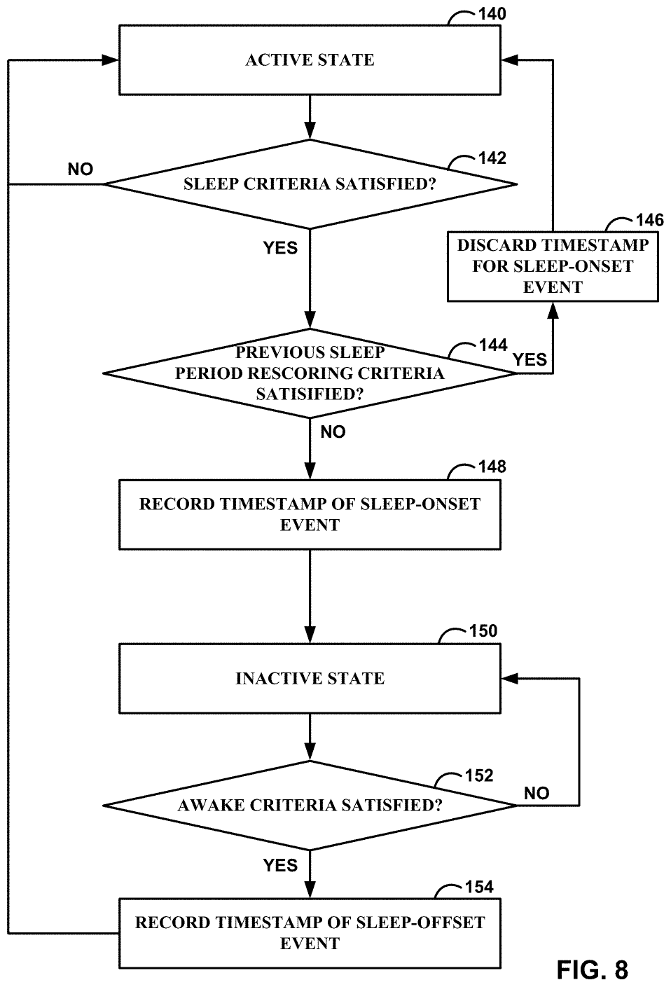

FIG. 8 is an example state diagram for identifying transitions between inactive state and active state based on activity data over time, in accordance with one or more examples of the present disclosure. When monitoring the patient's activity data (e.g., during a pre-configured portion of the patient's day), IMD 10 may detect a sleep-onset event transitioning the patient from active state to inactive state and/or an out-of-bed event transitioning the patient from inactive state to active state. According to the illustrated example of FIG. 8, processing circuitry 50 of IMD 10 determines one or more transitions from inactive state to active state or vice versa based on patient activity data generated by sensing circuitry 52 of IMD 10. Processing circuitry 50 of IMD 10 may operate in real-time or near real-time by applying a detection analysis for changes in patient health after a suitable amount of activity count classifications are recorded.

To commence such a detection analysis in the example operation of FIG. 7, processing circuitry 50 of IMD 10 processes patient activity data during a first time period (e.g., a half hour) that the patient is known to be in the active state (140). The active state may be initial starting point for the detection analysis, or the detection analysis may return to the active state after transitioning from an inactive state as demonstrated further below.

Processing circuitry 50 proceeds to determine whether the patient activity data satisfies the sleep criteria (142). As described herein, processing circuitry 50 of IMD 10 determines a number of activity count classifications for a number of intervals that encompass a timeslot and for each timeslot and records a corresponding number of activity count classifications in a dataset of the patient activity data (which may be stored in a memory buffer). Processing circuitry 50 may establish a maximum size for the dataset translating to one half-hour or 30 minutes of activity count classifications for comparing with the sleep monitoring criteria. It is possible that only a portion of the 30 minutes will be used for the comparison with the sleep criteria.

To illustrate by way of example sleep criteria, processing circuitry 50 may analyze the patient activity in the dataset to determine whether a) an oldest timeslot corresponds to zero (0) activity count classifications, b) a sum of ten oldest timeslots is less than two (2), and c) a sum of activity count classifications for all timeslots is less than five (5) between 12 am and 4 am or less than thirteen (13) otherwise. The combination of sleep criterion a, b, and c is configured to accurately determine if the patient has fallen asleep. If processing circuitry 50 determines that the patient activity data (e.g., the recorded activity count classifications) in the dataset satisfies the three sleep criterion a, b, and c, the patient's is sufficiently inactive for processing circuitry 50 to register a sleep-onset event transitioning between the active state to the inactive state at a beginning of the dataset. On the other hand, if the sleep criterion a, b, and c are not satisfied, the patient is mostly likely too active to be asleep.

Based on a determination that the patient activity data does not satisfy the sleep criteria (NO of 142), processing circuitry 50 does not register a transition to the inactive state and returns to processing more patient activity data while the patient is in the active state (140). Processing circuitry 50 may update the dataset of the patient activity data by recording one minute of 2-second ICs, shifting the dataset of recorded 2-second ICs for a previous thirty minutes by one minute, deleting a data entry recording an oldest latest minute of 2 second ICs (e.g., at the end of the dataset), and storing a data entry for the recent minute of patient activity data (e.g., at a beginning of the dataset). Processing circuitry 50 proceeds to apply the example sleep criteria, such as the above three sleep criterion a, b, and c, to the updated dataset of the patient activity data. Based on a determination that the updated dataset of patient activity data satisfies the example sleep criteria and indicates a sleep-onset event (YES of 142), processing circuitry 50 proceeds to verify the sleep-onset event by determining whether the dataset of the patient activity data satisfies previous sleep period rescoring criteria (144). Based on the previous sleep period rescoring criteria, processing circuitry 50 may determine that the dataset of the patient activity data has been misclassified as the sleep-onset event.

To illustrate by way of example previous sleep period rescoring criteria, processing circuitry 50 processes timestamps associated with recorded activity counts (e.g., ICs) and disqualifies the sleep-onset event as indicative of an actual transition to the inactive state if one or more of the following criteria are verified: a) an awake period between a previous sleep-offset/out-of-bed event and the sleep-onset event was greater than 30 minutes and a previous sleep period duration was less than one-hundred and twenty (120) minutes; and b) If the prior sleep period duration was less than sixty (60) minutes and the prior sleep-onset event occurred before 11 pm or after 6 am. The awake period may refer to an amount of time between the out-of-bed/sleep-offset event indicating a transition to the active state and the sleep-onset event.

Based on a determination that the patient activity data satisfies the previous sleep period rescoring criteria and the sleep-onset event is to be disqualified (YES of 144), processing circuitry 50 discards a timestamp for the sleep-onset event, recategorizes the dataset of patient activity data as a misclassified rest period, reclassifies the patient as being in the active state (146), and then, returns to process additional patient activity data while the patient is in the active state (140). Processing circuitry 50 may update the dataset of the patient activity data by recording a next minute of 2-second ICs, shifting the dataset of recorded 2-second ICs for a previous thirty minutes by one minute, deleting a data entry recording an oldest minute of 2 second ICs (e.g., at the end of the dataset), and storing a data entry for the recent minute of patient activity data (e.g., at a beginning of the dataset). After the update, processing circuitry 50 determines whether the updated dataset of the patient activity data satisfies the example sleep criteria and indicates a transition from active state to inactive state (142).

Processing circuitry 50 records a timestamp of the sleep-onset event as a valid transition to inactive state (148) based on a determination that the patient activity data does not satisfy the previous sleep period rescoring criteria (NO of 144). Processing circuitry 50 processes the patient activity data during a second time period when the patient in the inactive state (150). When a sufficient amount of patient activity data is recorded (e.g., three minutes to thirty minutes), processing circuitry 50 renders a determination as to whether the patient activity data satisfies awake criteria during the second time period (152). Based on a determination that the patient activity data does not satisfy the awake criteria (NO of 152), processing circuitry 50 returns to process additional patient activity data while the patient is in the inactive state (150). Based on a determination that the dataset of the patient activity data satisfies the awake criteria and indicates an out-of-bed/sleep-offset event (YES of 152), processing circuitry 50 proceeds to record a timestamp of out-of-bed/sleep-offset event as a valid transition from the inactive state to the active state (154) and then, returns to process additional patient activity data while the patient is in the active state (140).

To illustrate by way of example awake criterion, processing circuitry 50 may analyze the patient activity data in the buffer to determine whether a sum of 2-second ICs for a configurable number of minutes (e.g., one (1) or three (3)) exceeds a threshold activity count. Example awake criteria may include threshold activity counts for both one minute and three minute activity count sums. The example awake criteria may be partitioned into one or more criterion for detecting an onset of an out-of-bed event and one or more criterion for detecting an end of that out-of-bed event.

The following awake criteria includes one-minute and three-minute threshold activity counts for the out-of-bed event onset (e.g., sleep-offset timeslot) and separate one minute and three minute threshold activity counts for the out-of-bed event end (e.g., sleep end timeslot). In some examples, during a sleep period while the patient is in the inactive state, processing circuitry 50 applies the respective out-of-bed event onset threshold activity counts for (current) 1-minute periods (e.g., 50) and 3-minute periods (e.g., 150). After detecting satisfaction of both the 1-minute and 3-minute threshold activity counts within a same 3-minute period, processing circuitry 50 applies the respective out-of-bed event end threshold activity counts for (current) 1-minute periods (e.g., 50) and 3-minute periods (e.g., 230). Satisfaction of both of the above threshold activity counts should be contemporaneous. The above 1-minute thresholds enable localization of event onset and event end to specific 1-minute periods. Another criterion (e.g., a “time since out of bed” counter) may be used to post-process sleep windows (e.g., ensure that sleep onsets do not occur prior to out-of-bed events).

Processing circuitry 50 maintains the dataset to record, for each of at least the configurable number (e.g., thirty) of minutes, a total activity count for that minute, as described herein. If processing circuitry 50 determines that the most recent or newest three (3) minutes of activity counts in the dataset (in total) exceed the threshold activity count set to 150, the patient activity data (e.g., the recorded activity count or activity count classifications) in the dataset satisfies the above example awake criterion. Processing circuitry 50 may compare most recent or newest minute of activity counts (or activity count classifications) to a threshold as a second awake criterion (i.e., the one-minute threshold). Satisfaction of the second awake criterion localizes a precise time of the sleep-offset or the onset of out-of-bed event. Processing circuitry 50 may add another minute of activity counts to the dataset to localize a precise time of the sleep-offset or the onset of out-of-bed event. Therefore, the patient is sufficiently active for processing circuitry 50 to register an out-of-bed event (e.g., sleep-offset event) onset transitioning the patient from the inactive state to the active state.

After updating the dataset for at least a configured number of minutes (e.g., one or three), processing circuitry 50 applies the example awake criteria to the updated dataset to identify an end to the out-of-bed event and start of the patient's awake period. When processing circuitry 50 determines that the most recent or newest one (1) minute of activity counts in the buffer exceed the 1-minute threshold activity count set to 50, the patient activity data (e.g., the recorded activity count or activity count classifications) in the buffer satisfies the example awake criterion. Sequentially or concurrently to the above determination, processing circuitry 50 proceeds to determine that the most recent or newest three (3) minutes of activity counts in the buffer (in total) exceed the 3-minute threshold activity count set to 230. Satisfaction of both threshold activity counts with the updated buffer indicates that the patient activity data satisfaction of the awake criteria transitioning the patient from sleep period to awake period. Non-satisfaction causes the buffer to be further updated with a next minute of patient activity data (e.g., activity counts or activity count classifications) and reapplication of the above awake criteria.

In some examples, after detecting the onset of the out-of-bed event/sleep-offset event, processing circuitry 50 monitors recent patient activity data for new activity counts and/or activity count classifications, and if a time-since-out-of-bed counter elapses (e.g., runs past a predetermined length of time), processing circuitry 50 categorizes a corresponding timestamp with the onset of out-of-bed event. Processing circuitry 50 may require an end of the out-of-bed event to trigger an end of inactive state. Crossing the one-minute threshold may trigger temporary (e.g., volatile) storage of sleep-onset timestamps and invoke the time-since-out-of-bed counter as another awake criterion. When a current time crosses the time-since-out-of-bed-counter, processing circuitry 50 may be prompted to align recorded timestamps (e.g., to ensure that sleep-onset events do not occur prior to out-of-bed events). Crossing the three-minute threshold for the above example awake criterion may trigger permanent (e.g., non-volatile) storage of sleep-onset event timestamps. Sleep onset detection is disabled detection of the out-of-bed event.

Processing circuitry 50 may apply additional criterion even though the above awake criteria adequately identify transitions to the active state. As an option, processing circuitry 50 may apply a posture-related criterion to a current one-minute timeslot or a previous one-minute timeslot; to illustrate by way of an example optional criterion, if posture data (e.g., a posture measurement, such as an angle to upright reference) is less than or equal to 30 degrees, then the recent sampled timestamp is flagged and attributed with a start of an out-of-bed event. Once the out-of-bed event is detected, processing circuitry 50 may monitor patient activity data for low activity counts or activity count classifications every 10 seconds for a length another optional criterion. If the posture measurement (e.g., the posture angle to upright reference) is greater than 55 degrees, then the recent sampled timestamp is flagged as the end of the out-of-bed event. The above optional criterion may be invoked for a number of reasons, as an example, an activity count sum for a current timeslot or a previous time slot exceeding a threshold of 16 counts. If an activity count sum for a current timeslot or a previous time slot exceeds or equals a threshold of 16 counts, processing circuitry 50 may be instructed to apply the optional criterion.

Processing circuitry 50 may adjust an established length of a timeslot and/or employ overlapping timeslots. For example, processing circuitry 50 may compute activity counts for overlapping one-minute timeslots and use each respective activity for different activity count classification quantities. Processing circuitry 50 may dynamically adjust any of the above criteria to improve upon accuracy in identifying transitions between inactive states and active states and accurately recording timestamps and other data that correlates with these identified transitions.

Processing circuitry 50 may operate to perform the example operations illustrated in FIGS. 6-8 at pre-configured time periods over a day or a number of days. IMD 10 may be programmed with the pre-configured time period and operate when the patient normally falls asleep. As an alternative, IMD 10 may be programmed to operate at an arbitrary (e.g., selected at random) time period. Each time period may be one hour in length or multiple hours in length. Processing circuitry 50 may be programmed to use ten second time intervals but, in some examples, processing circuitry 50 may enlarge or shorten a length of the ten second interval, for instance, to improve upon the accuracy at which IMD 10 identifies transitions between inactive state and active state.

There are number of mechanisms for determining the number of activity count classifications in a time slot (e.g., a minute) such as those described herein of which one checks whether integrated counts of a frontal (z-axis) accelerometer reaches or exceeds a noise floor threshold within each of a number (e.g., six) of consecutive 10-second intervals. Processing circuitry 50 of IMD 10 may apply the example mechanism to each minute in the half-hour time period to determine a number of activity count classifications (e.g., with a one-minute resolution)) for that half-hour time period. After applying either sleep criteria or awake criteria, processing circuitry 50 of IMD 10 determines whether the patient transitioned between states during the half-hour time period. Processing circuitry 50 of IMD 10 may proceed to select (e.g., consecutive, latest, and/or oldest) time slots for the comparison with the sleep or awake criteria.

Depending upon which sleep quality metrics processing circuitry 50 of IMD 10 employs for the computation, the metric value also provides certain insights into the patient sleep activities and sleep quality. An example metric may refer to mathematical functions (e.g., formulas), data structures (e.g., models), standardized methods/mechanisms, and measurements and other data configured to enable the computation of accurate metric values by way of determining when a patient is awake (i.e., active state) or asleep (i.e., inactive state). Such an analysis may be qualitative and/or quantitative, for example, to gain insight into the patient's health by expressing as many related features as possible given the context of patient sleep activity.

By relying upon these time periods (and possibly disregarding other time periods), the techniques described herein enable a highly accurate assessment of patient health of which acute changes are easily detected. Sleep quality metric values computed from inactive state activity data and/or active state activity data indicate an overall sleep quality for trending and long-term analysis. An abrupt increase or decrease in sleep restlessness or an abrupt decrease in night-time activity could indicate an acute change in health. Each activity metric as described herein may be configured to quantify some aspect of patient activity, such that combining one or more metrics may provide a holistic view or assessment of patient health.

The order and flow of the operation illustrated in FIGS. 6, 7, and 8 are examples. In other examples according to this disclosure, more or fewer thresholds may be considered. Further, in some examples, processing circuitry may perform or not perform the methods of FIG. 6, FIG. 7, and FIG. 8, or any of the techniques described herein, as directed by a user, e.g., via external device 12 or computing devices 100. For example, a patient, clinician, or other user may turn on or off functionality for identifying changes in patient health (e.g., using Wi-Fi or cellular services) or locally (e.g., using an application provided on a patient's cellular phone or using a medical device programmer).

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. A medical system comprising:
one or more sensors configured to sense patient activity;
sensing circuitry configured to provide patient activity data based on the sensed patient activity; and
processing circuitry configured to:
determine, from the patient activity data, for each of a plurality of intervals, a respective activity classification, wherein each activity classification indicates whether the patient activity data during the interval satisfies at least one predetermined criterion indicative of patient movement;
for each of a plurality of timeslots, determine a number of intervals that satisfy the at least one predetermined criterion, each timeslot including a consecutive subset of the plurality of intervals; and
identify transitions between an inactive state of the patient and an active state of the patient based on the determined numbers of intervals within the plurality of timeslots.

2. The medical system of claim 1, wherein the processing circuitry is further configured to:
determine a sleep quality metric value based on the identified transitions between the inactive state of the patient and the active state of the patient; and
compare the sleep quality metric value to a patient health threshold.

3. The medical system of claim 1, wherein to identify the transitions between the active state and the inactive state, the processing circuitry is configured to:
while a current state of the patient activity data comprises the active state, perform a first set of operations comprising:
determining, from the patient activity data, a first set of activity count classifications for a first consecutive subset of the plurality of timeslots encompassing a first time period, wherein each timeslot corresponds to a number of activity count classifications; and
in response to determining a sleep-onset event based upon an application of sleep criteria to a portion of the first set of activity count classifications, setting the current state to comprise the inactive state and performing a second set of operations; and
while the current state of the patient activity data comprises the inactive state, perform the second set of operations comprising:
determining, from the patient activity data, a second set of activity count classifications for a second consecutive subset of the plurality of timeslots encompassing a second time period, wherein each timeslot corresponds to a number of activity count classifications, and
in response to determining an out-of-bed event based upon an application of awake criteria to a portion of the second set of activity count classifications, setting the current state to the active state and performing the first set of operations.

4. The medical system of claim 1, wherein to identify a transition from the active state to the inactive state, the processing circuitry is configured to:
determine, from the patient activity data, a first set of activity count classifications for a first consecutive subset of the plurality of timeslots encompassing a first time period, wherein each timeslot corresponds to a number of activity count classifications; and
in response to determining a sleep-onset event based upon an application of sleep criteria to a portion of the first set of activity count classifications, set the current state to comprise the inactive state.

5. The medical system of claim 1, wherein to identify a transition from the inactive state to the active state, the processing circuitry is configured to:
determine, from the patient activity data, a first set of activity count classifications for a first consecutive subset of the plurality of timeslots encompassing a first time period, wherein each timeslot corresponds to a number of activity count classifications, and
in response to determining an onset and an end of an out-of-bed event based upon an application of awake criteria to a portion of the first set of activity count classifications, set the current state to the active state.

6. The medical system of claim 1, wherein to determine, from the patient activity data, the activity count classifications, the processing circuitry is configured to:
for each time slot of a time period,
for each interval in that timeslot,
compare a corresponding activity count to a noise floor to determine whether to that interval corresponds to a positive activity count classification.

7. The medical system of claim 6, wherein to determine, from the patient activity data, for each of a plurality of intervals, a respective activity count classification, the processing circuitry is further configured to: determine an activity count for each hour in a twenty-four hour time period and select, for a noise floor, a highest activity count amongst a pre-configured number of lowest activity counts.

8. The medical system of claim 1, wherein the processing circuitry is configured to identify the transitions during a pre-determined portion of any given day.

9. The medical system of claim 1 further comprising a medical device that includes the one or more sensors configured to sense the patient activity, wherein the medical device comprises at least one of an implantable device, a wearable device, a cardiac monitor, a pacemaker/defibrillator, or a ventricular assist device (VAD) that comprises the one or more sensors and the sensing circuitry.

10. The medical system of claim 1, wherein to determine, from the patient activity data, for each of a plurality of intervals, a respective activity count classification, the processing circuitry is further configured to: compare a current transition between the inactive state and the active state to rescoring criteria to determine whether to discard any data for a previous transition between the inactive state and the active state.

11. The medical system of claim 1 further comprising a storage device that include a buffer for storing the patient activity data as a dataset,
wherein the processing circuitry is further configured to delete an oldest data entry comprising patient activity data, shift the dataset by one interval, and store patient activity data for a recent interval.

12. The medical system of claim 11, wherein the buffer is configured with a maximum size for storing the patient activity data.

13. A method, comprising:
sensing patient activity via one or more sensors;
generating, via sensing circuitry, patient activity data based on the sensed patient activity;
from the patient activity data, determining, by processing circuitry, for each of a plurality of intervals, a respective activity classification, wherein each activity classification indicates whether the activity data during the interval satisfies at least one predetermined criterion indicative of patient movement;
for each of a plurality of timeslots, determining, by the processing circuitry, a number of intervals that satisfy the at least one predetermined criterion, each timeslot including a consecutive subset of the plurality of intervals; and
identifying, by the processing circuitry, transitions between an inactive state of the patient and an active state of the patient based on determined numbers of intervals within the plurality of timeslots.

14. The method of claim 13, wherein identifying the transitions further comprises: for each identified transition, comparing data corresponding to the corresponding activity count classifications within the plurality of timeslots to a sleep quality metric to produce a sleep quality metric value; and compare the sleep quality metric value to a patient health threshold.

15. The method of claim 13, wherein identifying a transition from the active state to the active state further comprises:
while the current state of the patient activity data comprises the inactive state,
determining, from the patient activity data, at least one set of activity count classifications for at least one consecutive subset of the plurality of timeslots encompassing a time period, wherein each timeslot corresponds to a number of activity count classifications, and
in response to determining a sleep-onset event based upon an application of sleep criteria to a portion of the first set of activity count classifications, setting the current state to comprise the inactive state.

16. The method of claim 13, wherein identifying a transition from the inactive state to the active state further comprises:
while the current state of the patient activity data comprises the inactive state,
determining, from the patient activity data, a first set of activity count classifications for a first consecutive subset of the plurality of timeslots encompassing a first time period, wherein each timeslot corresponds to a number of activity count classifications, and
in response to determining an onset and an end of an out-of-bed event based upon an application of awake criteria to a portion of the first set of activity count classifications, setting the current state to the active state.

17. The method of claim 13, wherein determining, from the patient activity data, for each of a plurality of intervals, the respective activity count classification further comprises: for each timeslot of the plurality of timeslots, determining a number of activity count classifications based a numbers of intervals within the timeslot satisfying a noise floor.

18. The method of claim 17, further comprising determining an activity count for each hour in a twenty-four hour time period and selecting, for the noise floor, a highest activity count amongst a pre-configured number of lowest activity counts.

19. The method of claim 13, wherein an implantable device or a wearable device includes the one or more sensors configured to sense the patient activity.

20. A non-transitory computer-readable storage medium comprising program instructions that, when executed by processing circuitry of a medical system, cause the processing circuitry to:
sense patient activity via one or more sensors;
generate, via sensing circuitry, patient activity data based on the sensed patient activity;
determine, from the patient activity data, for each of a plurality of intervals, a respective activity classification, wherein each activity classification indicates whether the activity data during the interval satisfies at least one predetermined criterion indicative of patient movement;
for each of a plurality of timeslots, determine a number of intervals that satisfy the at least one predetermined criterion, each timeslot including a consecutive subset of the plurality of intervals; and
identify transitions between an inactive state of the patient and an active state of the patient based on determined numbers of intervals within the plurality of timeslots.
